(19) United States
(12) Patent Application Publication
(10) Pub. No.: US 2026/0257006 A1
(43) Pub. Date: Sep. 3, 2026

(54) CONTAINER FOR PACKAGING PRE-FILLED SYRINGE

(71) Applicant: SAM CHUN DANG PHARM. CO., LTD., Hwaseong-si (KR)

(72) Inventors: Byung Jhip HA, Hwaseong-si (KR); Chang Hoon YOO, Hwaseong-si (KR); Gyeong Won CHOI, Hwaseong-si (KR); Seon Jin KIM, Hwaseong-si (KR); Dong Kyu KIM, Hwaseong-si (KR)

(21) Appl. No.: 19/489,229

(22) PCT Filed: Mar. 20, 2024
(86) PCT No.: PCT/KR2024/003518

§ 371 Date: Dec. 1, 2025

(30) Foreign Application Priority Data
May 31, 2023 (KR) 10-2023-0070232

(51) Int. Cl. A61M 5/00; B65D 75/36

(52) U.S. Cl. CPC A61M 5/002; B65D 75/366

(57) ABSTRACT

Provided is a blister pack of a syringe, comprising: a container body including a receiving part containing a space molded to accommodate a pre-filled syringe; a fixing part protruding to fix a portion of the syringe accommodated in the receiving part; and a sealing part to which a sealing film is attachable, wherein the fixing part includes a second fixing part and optionally further includes a first fixing part, the receiving part is separated by the fixing part to form at least any one of a first receiving part and a second receiving part, and a third receiving part, and when the syringe is accommodated in the receiving part of the blister pack, a movable distance of a plunger of the syringe is less than or equal to a spacing distance between ribs included in a stopper.

### TECHNICAL FIELD

The present invention relates to a blister pack for packaging a pre-filled syringe, and more particularly to a blister pack comprising a pre-filled syringe that is suitable for preventing breakage during transportation and maintaining sterilization.

### BACKGROUND ART

A pre-filled syringe is a preloaded syringe distributed with drug solution already filled in the syringe, and is gaining traction due to reduction of syringe reuse and bloodborne pathogen contamination, both of which are problems when administering syringe vials.

The pre-filled syringe is typically packaged in a blister container, but in some cases, if the container is not sufficiently secured and excessive vibration occurs during transportation, the drug solution in the syringe may vibrate and leak during transportation, especially in the case of vibration-sensitive drug solutions, e.g. protein solutions, causing deterioration or precipitation.

In addition, the pre-filled syringe is sterilized using a final sterilization method in which the syringe is sterilized with vaporized hydrogen peroxide or ethylene oxide gas under reduced pressure conditions after filling the drug solution into the syringe and sealing it so that the entire syringe is hermetically packaged in packaging such as a blister pack. However, currently, during the sterilization process under reduced pressure conditions such as ultra-low pressure, for example, 10 mbar, air bubbles generated in the syringe when filling the drug solution expand under reduced pressure, causing the cap or handle of the discharge port to move, risking the integrity of the packaging and potential exposure of the drug solution, ultimately resulting in syringe damage.

Against this background, the present inventors have developed a blister pack for packaging a pre-filled syringe capable of preventing a syringe or a plunger from retracting or advancing longitudinally and preventing breakage during external impacts such as transport or falling or during a sterilization process inside the blister pack.

### DISCLOSURE OF INVENTION

### Technical Problem

An object of the present invention is to provide a blister pack of a syringe, comprising: a container body 71 including a receiving part 72 containing a space molded to accommodate a pre-filled syringe; a fixing part 75 protruding to fix a portion of the syringe accommodated in the receiving part 72; and a sealing part 73 to which a sealing film 74 is attachable, wherein the fixing part 75 includes a first fixing part 75a and a second fixing part 75b, the receiving part 72 is separated by the fixing part 75 to form a first receiving part 72a, a second receiving part 72b, and a third receiving part 72c, and when the syringe is accommodated in the receiving part of the blister pack, a movable distance of a plunger 4 of the syringe is less than or equal to a spacing distance between ribs included in a stopper 10.

### Solution to Problem

In one general aspect, there is provided a blister pack of a syringe, comprising: a container body 71 including a receiving part 72 containing a space molded to accommodate a pre-filled syringe; a fixing part 75 protruding to fix a portion of the syringe accommodated in the receiving part 72; and a sealing part 73 to which a sealing film 74 is attachable,
wherein the fixing part 75 includes a second fixing part 75b and optionally further includes a first fixing part 75a,
the receiving part 72 is separated by the fixing part 75 to form at least any one of a first receiving part 72a and a second receiving part 72b, and a third receiving part 72c, and
when the syringe is accommodated in the receiving part of the blister pack, a movable distance of a plunger 4 of the syringe is less than or equal to a spacing distance between ribs included in a stopper 10.

The fixing part 75 may comprise the first fixing part 75a and the second fixing part 75b, and the receiving part 72 may comprise the first receiving part 72a and the second receiving part 72b separately formed by the first fixing part 75a, and may comprise the second receiving part 72b and the third receiving part 72c separately formed by the second fixing part 75b.

The fixing part 75 may comprise the second fixing part 75b, and may comprise the second receiving part 72b and the third receiving part 72c separately formed by the second fixing part 75b.

The blister pack may have a pre-filled syringe accommodated in the receiving part.

The spacing distance between the ribs included in the stopper 10 may be 1 to 4 mm.

When the syringe is accommodated in the receiving part of the blister pack, a spacing between a disk-shaped flange 42 at a rear part of the syringe and a facing wall of the third receiving part 72c may be 4 mm or less.

The container body 71 constituting the blister pack 7 may be formed of a plastic film or sheet having rigid properties in order to fix and maintain the accommodated syringe.

The container body 71 may be formed of one or more plastic films or sheets selected from the group consisting of polyvinyl chloride (PVC), polyethylene terephthalate (PET), polypropylene (PP), polystyrene, polyethylene, polyester, and polypropylene.

The syringe may be received in the receiving part so that a portion of the body 2 is interlocked by the fixing part 75, and the fixing part 75 may protrude in an inward direction of the container body so that a portion of the body 2 of the syringe is fixed in a tightening manner by the fixing part 75.

The first receiving part 72a may receive a portion of an upper part of the syringe, including a sealing part 8 of the syringe, the second receiving part 72b may receive a central part of the syringe, including the portion of the body 2 of the syringe, and the third receiving part 72c may receive a rear part of the plunger, including a finger grip part 6 and a disk-shaped flange 42 of the syringe.

When the fixing part 75 includes the second fixing part 75b alone, the second receiving part 72b may receive the upper part of the syringe, including the seal part 8 of the syringe, and the central part of the syringe, including a portion of the body 2 of the syringe, and the third receiving part 72c may receive the rear part of the plunger, including the finger grip part 6 and the disk-shaped flange 42 of the syringe.

In the blister pack of a syringe, a wall of the third receiving part 72c facing the disk-shaped flange 42 may support the disk-shaped flange 42 of the plunger, thereby preventing the syringe or plunger from retracting and moving in a second axis B longitudinal direction. The disc-shaped flange 42 may be in contact with a wall of the third receiving part 72c facing the disc-shaped flange 42.

The syringe may not include a retraction prevention part.

When the syringe is received in the blister pack, a variable volume chamber 18 of the syringe may have a maximum volume.

The blister pack 7 may comprise a sealing film 74 adhered to the sealing part 73, and the receiving part may be sealed by adhesion of the sealing part 73 to the sealing film 74.

The sealing part 73 may comprise an adhesive member so that one side of the sealing film 74 is attached.

The sealing film 74 may be detachably adhered to the sealing part 73.

The syringe may comprise: a body 2 having an outlet 12 at an outlet end 14; a plunger 4 located inside the body 2; and a stopper 10 which is located at a front part of the plunger 4 inside the body 2, and includes a front surface 16 forming a variable volume chamber 18 with the body 2 to discharge an ophthalmic formulation 20, which is filled into the variable volume chamber 18, through the outlet 12 by movement of the plunger 4, wherein the plunger 4 comprises a plunger rod 41 extending in a longitudinal direction, and a flange part 22 located at an end 24 of the plunger rod, which is adjacent to the stopper 10.

The syringe may comprise an ophthalmic formulation.

The ophthalmic formulation may be used to administer to patients with ophthalmic diseases.

### Advantageous Effects of Invention

The blister pack of the pre-filled syringe according to the present invention may prevent breakage and damage by stably fixing the pre-filled syringe under high-pressure or low-pressure conditions, when external shock is applied, or even during the sterilization process.

Furthermore, the blister pack of the pre-filled syringe according to the present invention may be completely sterilized and may prevent secondary infection by bacteria, etc., during use or storage post-sterilization.

### BRIEF DESCRIPTION OF DRAWINGS

FIG. 1 is a perspective view of a side of a blister pack containing a syringe.

FIG. 2 is a perspective view of a front surface of the blister pack containing a syringe.

FIG. 3 is a perspective view of the blister pack of a syringe of the present invention.

FIG. 4 is a perspective view of the blister pack containing a pre-filled syringe.

FIG. 5 is a perspective view of the side of the blister pack containing a syringe.

FIG. 6 is a perspective view of the blister pack of a syringe of the present invention.

FIG. 7 is a perspective view of the blister pack containing a pre-filled syringe.

FIG. 8 shows the syringe.

FIGS. 9 and 10 are perspective views of the side of the syringe.

FIGS. 11, 12, and 13 show a plunger of the syringe.

FIG. 14 is a cross-sectional view of a plunger rod.

FIG. 15 is a cross-sectional view of a stopper.

FIGS. 16 and 17 show cross-sections of the stopper and the coupling between the plunger and stopper.

FIG. 18 is a view showing a side and a cross-section of a seal part.

FIG. 19 shows a finger grip part.

### BEST MODE FOR CARRYING OUT THE INVENTION

The present invention provides a blister pack comprising a pre-filled syringe. The pre-filled syringe may be filled with an ophthalmic formulation. The syringe is intended to be used for administration to a patient with an ocular disease, wherein the ocular disease may comprise visual impairment due to age-related macular degeneration (AMD), diabetic macular edema (DME), episodic patchy edema of retinal vein occlusion (branch RVO or central RVO), diabetic retinopathy in patients with diabetic macular edema, or visual impairment due to choroidal neovascularization (CNV) secondary to pathological myopia.

Hereinafter, the present invention will be described in more detail.

### Blister Pack of Syringe Comprising Pre-Filled Syringe

In an aspect of the present invention, there is provided a blister pack of a syringe, comprising: a container body 71 including a receiving part 72 containing a space molded to accommodate a pre-filled syringe; a fixing part 75 protruding to fix a portion of the syringe accommodated in the receiving part 72; and a sealing part 73 to which a sealing film 74 is attachable,
wherein the fixing part 75 includes a second fixing part 75b and optionally further includes a first fixing part 75a,
the receiving part 72 is separated by the fixing part 75 to form at least any one of a first receiving part 72a and a second receiving part 72b, and a third receiving part 72c, and
when the syringe is accommodated in the receiving part of the blister pack, a movable distance of a plunger 4 of the syringe is less than or equal to a spacing distance between ribs included in a stopper 10.

The blister pack of the syringe may contain a syringe pre-filled with a drug solution accommodated therein.

The pre-filled syringe may be a syringe comprising a body, a stopper, and a plunger, and containing a medical agent in the form of a fluid within a volume chamber, which is the volume space by the body and the stopper. The body may comprise an outlet capable of discharge the filled medical agent, and the plunger may be in the form of a long rod in contact with or combined with the stopper. The medical agent capable of pushing the filled medical agent to the outlet may be discharged by movement of the plunger with which the stopper is in contact or coupled. The syringes are not limited, and may include any syringe form conventionally used in the art.

Further, the medical agent herein may include an ophthalmic preparation, preferably a protein preparation, more preferably a liquid preparation of a VEGF-antagonist, and even more preferably a liquid preparation of Aflibercept, but the medical agent herein is not limited thereto in scope, and may include a wide range of general liquid preparations capable of being administered by syringe.

Since the pre-filled syringe contains the medical agent in the form of a fluid, it is necessary to ensure that the syringe or the plunger of the syringe does not retract or advance longitudinally during transport and movement or sterilization.

Accordingly, the present invention implemented a blister pack for packaging a pre-filled syringe in which the pre-filled syringe is fixedly received and the space in which the syringe is receivable is designed to fit the length of the syringe, so that the syringe and/or the plunger of the syringe does not retract or advance even during a decompression sterilization process, and breakage is prevented.

In the blister pack of the syringe, the fixing part 75 includes the second fixing part 75b and optionally further includes the first fixing part 75a, which indicates that the fixing part 75 may include both the fixing part 75b and the first fixing part 75a, or the fixing part 75 may include the second fixing part 75b alone.

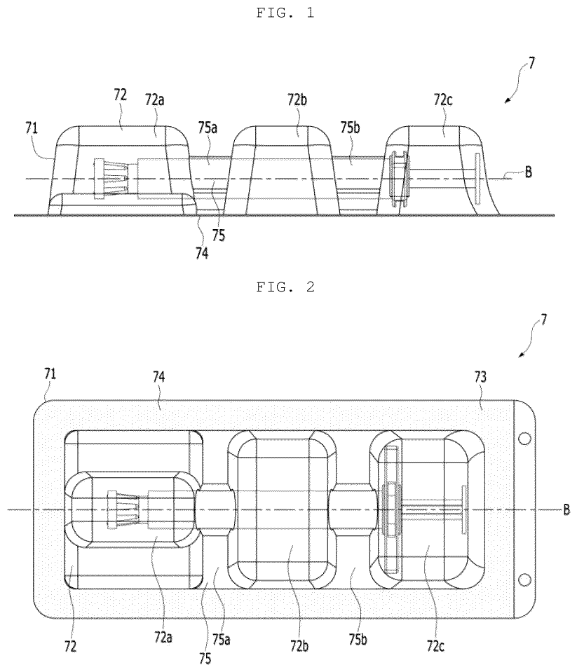

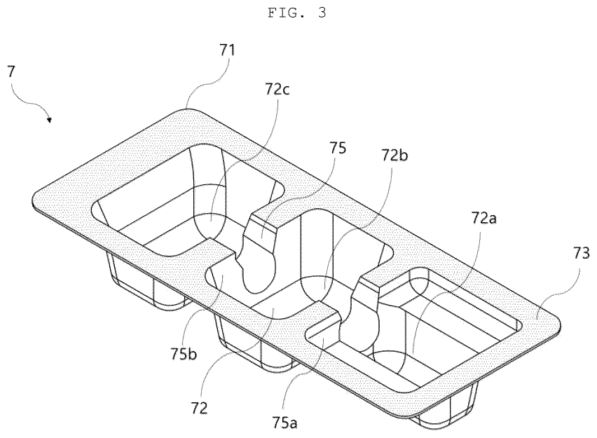

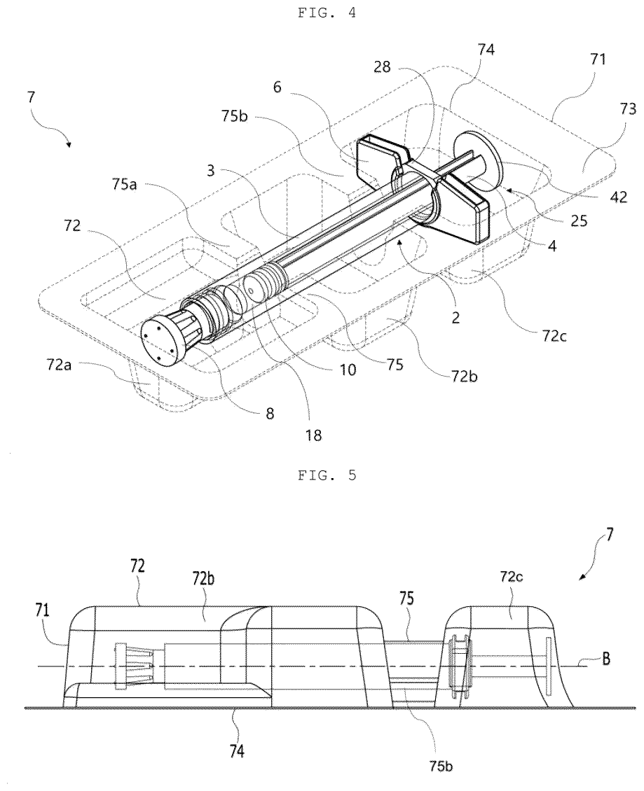

In an embodiment, the fixing part 75 may comprise the first fixing part 75a and the second fixing part 75b, and the receiving part 72 may comprise the first receiving part 72a and the second receiving part 72b separately formed by the first fixing part 75a and may comprise the second receiving part 72b and the third receiving part 72c separately formed by the second fixing part 75b. As described above, the blister pack of the syringe comprising the first fixing part 75a and the second fixing part 75b is shown in FIGS. 1 to 4.

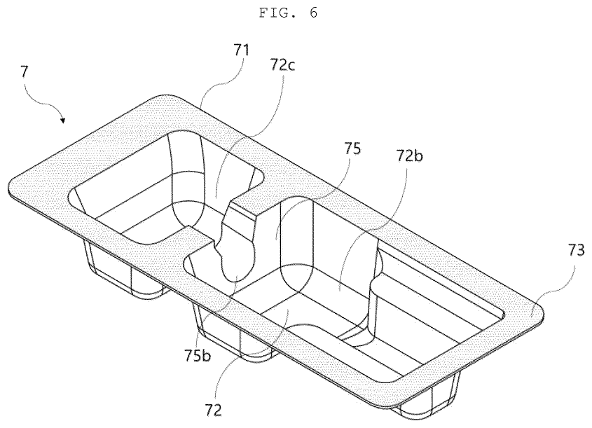

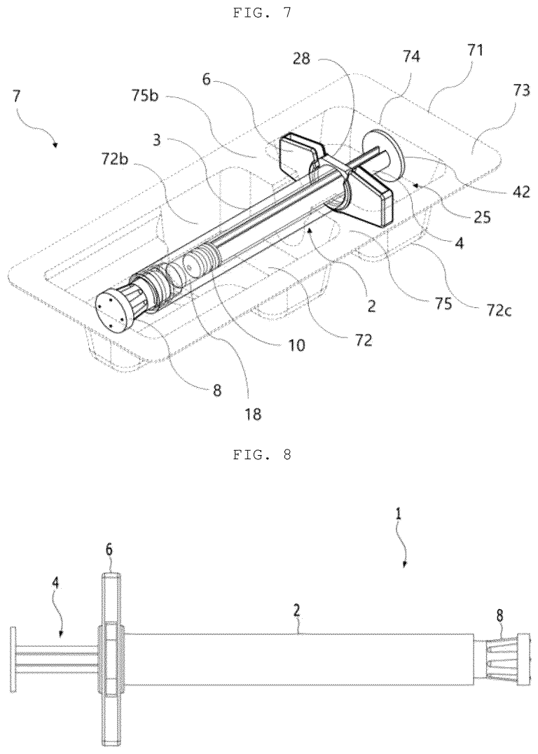

In an embodiment, the fixing part 75 may comprise the second fixing part 75b, and may comprise the second receiving part 72b and the third receiving part 72c separately formed by the second fixing part 75b. The blister pack of the syringe having this type is shown in FIGS. 5 through 7.

The movable distance of the plunger 4 may be a distance that the plunger 4 (specifically, a plunger rod 41) is movable in a second axis B longitudinal direction during reduced pressure sterilization to sterilize the inside of the blister pack. Specifically, the movable distance may be an advanceable distance (forward movable distance) or a retractable distance (backward movable distance). The advanceable distance may refer to a distance that the plunger 4 moves in the second axis B longitudinal direction, closer to an outlet end, and the retractable distance may refer to a distance that the plunger 4 moves in the second axis B longitudinal direction, away from the outlet end.

The spacing distance between ribs included in the stopper 10 refers to an axial spacing between adjacent ribs. In an embodiment, the spacing distance between the ribs, which is a spacing between adjacent ribs in at least two ribs 50, 51, 52 formed around an outer surface of the stopper, may be a spacing distance between the ribs 50, 51 or a spacing distance between the ribs 50, 52 when there are three ribs 50, 51, 52. Further, when there are two ribs 51, 52, the spacing distance may be a spacing distance between the ribs 51, 52. The spacing distance between the ribs may be 1 to 4 mm, and for example, a lower limit of the spacing distance may be 1.2 mm, 1.4 mm, 1.6 mm or 1.8 mm or more, and an upper limit thereof may be 3.8 mm, 3.6 mm, 3.4 mm, 3.2 mm, 3.0 mm, 2.8 mm, 2.6 mm, 2.4 mm or 2.2 mm or less.

In an embodiment, the movable distance of the plunger 4 of the syringe may be 4 mm, 3.8 mm, 3.6 mm, 3.4 mm, 3.2 mm, 3.0 mm, 2.8 mm, 2.6 mm, 2.4 mm, 2.2 mm, 2.0 mm, 1.8 mm, 1.6 mm, 1.4 mm, 1.2 mm, 1.0 mm, 0.8 mm, 0.6 mm, 0.4 mm, 0.2 mm or 0.1 mm or less, or may be 0 mm, which means that the plunger of the syringe does not move (advance or retract).

If the inside of the blister pack needs to be sterilized while the syringe is in place, when the air bubbles generated at the time of filling the syringe with drug solution during reduced pressure sterilization expand, the plunger may be retracted beyond the spacing distance between the stopper ribs, which may cause a problem that the filled drug solution comes into contact with non-sterile areas.

In an embodiment, the spacing distance between the ribs may be a distance between the peaks of the ribs, which is the highest portion of the ribs in substantial contact with an inner surface of the body of the syringe.

In an embodiment, the container body 71 constituting the blister pack 7 may be formed of a plastic film or sheet having rigid properties in order to fix and support the accommodated syringe. The rigid properties mean that the container body has a hardness such that a shape of the container body does not collapse even under an impact or external pressure that may occur in the event of movement and transportation or falling.

Further, the container body 71 may have a transparency such that the syringe 1 received in the container body is able to be seen from the outside.

The plastic film or sheet forming the container body 71 may include, but not limited to, polyvinyl chloride (PVC), polyethylene terephthalate (PET), polypropylene (PP), polystyrene, polyethylene, polyester, polypropylene, and the like, preferably polyethylene terephthalate (PET), and any material having transparency and rigid properties may be used to form the container body.

In the present invention, the syringe may be received in the receiving part so that a portion of the body 2 is interlocked by the fixing part 75, and the fixing part 75 may protrude in an inward direction of the container body so that a portion of the body 2 of the syringe is fixed in a tightening manner by the fixing part 75.

In an embodiment, the container body 71 may comprise the fixing part 75 that serves to fix the syringe, which is molded to fit a diameter of the syringe, enabling the interlocking and fixation of a portion of the received syringe, and the fixing part 75 may have a central concave shape to support the syringe. The fixing part 75 comprises the first fixing part 75a and the second fixing part 75b. The syringe is received in the receiving part and a portion of the body 2 is interlocked and fixed by the fixing part 75. The fixing part 75 may protrude in an inner direction of the container body so that the portion of the body 2 of the syringe is fixed in a tightening manner by the fixing part 75, and the fixing part 75 may function as a buffer to prevent damage to the syringe received in the blister pack from external impact or falling.

In an embodiment, when the fixing part 75 comprises the first fixing part 75a and the second fixing part 75b, the receiving part 72 may comprise the first receiving part 72a, the second receiving part 72b, and the third receiving part 72c, which are three receiving parts separately formed by the first fixing part 75a and the second fixing part 75b. Here, the first receiving part 72a may receive a portion of an upper part of the syringe, including a seal part 8 of the syringe, the second receiving part 72b may receive a central part of the syringe, including the portion of the body 2 of the syringe, and the third receiving part 72c may receive a rear part of the plunger, including a finger grip part 6 and a disk-shaped flange 42 of the syringe.

In an embodiment, when the fixing part 75 includes the second fixing part 75b alone, the receiving part 72 may comprise the second receiving part 72b and the third receiving part 72c, which are two receiving parts separately formed by the second fixing part 75b. Here, the second receiving part 72b may receive an upper part of the syringe, including the seal part 8 of the syringe, and a central part of the syringe, including a portion of the body 2 of the syringe, and the third receiving part 72c may receive a rear part of the plunger, including the finger grip part 6 and the disk-shaped flange 42 of the syringe.

The portion of the upper part of the syringe including the seal part 8 of the syringe may indicate a part, excluding a portion of the upper part of a body part 3 fixed by the first fixing part 75a among the upper parts of the syringe including the seal part 8, the outlet end 14, and a portion of the body part 3. The central part of the syringe received in the second receiving part 72b may indicate a portion of the body, excluding a portion of the first receiving part 72a and the third receiving part 72c of the syringe received therein and a portion of the syringe supported by the fixing part 75. The third receiving part 72c may receive the rear part of the plunger, including the finger grip part 6 and the disk-shaped flange 42 of the syringe.

The second fixing part 75a may fix or support a portion of the body of the syringe, excluding the rear part of the plunger including the finger grip part 6 and the disk-shaped flange 42 and a portion of the upper part of the syringe supported by the first fixing part 75a, and may preferably fix or support a portion of the body of the syringe adjacent to the finger grip part 6 so that the finger grip part 6 is received in the third receiving part 72c.

In the present invention, a wall of the third receiving part 72c facing the disk-shaped flange 42 may support the disk-shaped flange 42 of the plunger, thereby preventing the syringe or plunger from retracting and moving in the second axis B longitudinal direction.

In an embodiment, when the syringe is accommodated in the receiving part of the blister pack, a spacing between the disk-shaped flange 42 at the rear part of the syringe and the wall of the third receiving part 72c facing the disk-shaped flange may be 4 mm or less. Specifically, a spacing between the disk-shaped flange 42, which is the rear part of the plunger 4 of the syringe received inside the container body 71, and the wall of the third receiving part 72c facing the disk-shaped flange 42 may be 4 mm, 3.8 mm, 3.6 mm, 3.4 mm, 3.2 mm, 3 mm, 2.8 mm, 2.6 mm, 2.4 mm, 2.2 mm, 2 mm, 1.8 mm, 1.6 mm, 1.4 mm, 1.2 mm, 1 mm, 0.9 mm, 0.8 mm, 0.7 mm, 0.6 mm, 0.5 mm, 0.4 mm, 0.3 mm, 0.2 mm, 0.1 mm, 0.05 mm, 0.01 mm or less, preferably the disk-shaped flange 42 may be in contact with the wall of the third receiving part 72c facing the disc-shaped flange 42. Thus, the syringe is prevented from moving in a longitudinal direction or the plunger is prevented from retracting and moving in the second axis B longitudinal direction.

In an embodiment, the spacing between the seal part 8 of the syringe received inside the container body 71 and the wall of the first receiving part 72a facing the seal part 8 may be 2 cm, 1.5 cm, 1 cm, 5 mm, 4.5 mm, 4 mm, 3.5 mm, 3 mm, 2.5 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.05 mm, 0.01 mm or less, and spacing is not limited thereto, or the seal part 8 may be in contact with the wall of the first receiving part 72a facing in the longitudinal direction. Thus, the syringe is prevented from moving in the second axis a B longitudinal direction.

In the present invention, the syringe may not comprise a plunger shoulder formed on the plunger to prevent the plunger from disengagement. The plunger shoulder extends beyond the outer diameter of the plunger rod but is still embedded within the body to stabilize the movement of the plunger rod within the body by reducing the movement of the plunger rod perpendicular to the first axis A. However, even if the plunger of the syringe does not comprise the plunger shoulder, the blister pack of the present invention, implemented so that the distance the plunger may retract is limited to no more than the spacing distance between the ribs of the stopper of the syringe, may serve as a substitute for the plunger shoulder to prevent movement, such as retraction or advancement, of the plunger of the syringe and movement of the plunger rod during other operations or final sterilization operations in which the pressure may vary within or outside the variable volume chamber of the syringe and to maintain sterility.

When the syringe is received within the blister pack, a variable volume chamber 18 of the syringe may have a maximum volume, and more specifically, the variable volume chamber 18 of the syringe may have a maximum volume due to the inclusion of the medical agent in the form of a fluid within the variable volume chamber 18 of the syringe.

In the present invention, the blister pack 7 may comprise a sealing film 74 adhered to the sealing part 73, and the receiving part may be sealed by adhesion of the sealing part 73 and the sealing film 74.

The sealing part 73 may comprise an adhesive member so that one side of the sealing film 74 is attached. The adhesive member may be glue, paste, film, tape, pressure sensitive material, and/or low temperature adhesive, or the like, and may be any adhesive material capable of being adhered by heat or pressure such that the sealing film is peelable from the sealing part.

The sealing film 74 is detachably adhered to the sealing part 73 included in the container body 71, and may be paper, aluminum film, plastic film, plastic sheet, or plastic nonwoven fabric, and may preferably be a high-density polyethylene (HDPE) film. In an embodiment, a TYVEK®1073B product was used as the sealing film 74.

The blister pack of the present invention contains the syringe received in a suspended configuration, which prevents breakage due to impact such as falling or vibration during transportation, and the receiving part 72 is designed to fit the length of the syringe in the second axis B direction to limit movement of the syringe in the second axis B direction. Furthermore, the interior of the blister pack may be sterilized using vaporous hydrogen peroxide or ethylene oxide gas after the Tyvek lid is sealed. In detail, the blister pack of the present invention has the advantage of preventing the stopper from retracting while expanding under the conditions of reduced pressure accompanying the hydrogen peroxide sterilization process or the ethylene oxide gas sterilization process.

In an embodiment, the blister pack of the present invention may have a spacing of 1 cm or less between the received syringe and the sealing film 74, for example, 9 mm, 8 mm, 7 mm, or 6 mm or less, and the spacing may be 5±0.5 mm. However, the spacing is not limited to this, and a portion of the finger grip part 6 of the syringe may be in contact with the sealing film 74, and the spacing from the sealing film 74 may be different depending on each part of the syringe.

In an embodiment, the blister pack of the present invention may have a total length in the B-axis longitudinal direction of 100 to 150 mm, and specifically, the lower limit thereof may be 105 mm, 110 mm, or 115 mm or more, and the upper limit thereof may be 145 mm, 140 mm, 135 mm, 130 mm, or 125 mm or less, and preferably 120±0.5 mm.

In an embodiment, the blister pack of the present invention may have a total length in a direction perpendicular to the B-axis longitudinal direction of 40 to 60 mm, specifically, the lower limit thereof may be 42 mm, 44 mm, 46 mm, 48 mm, or 50 mm or more, and the upper limit thereof may be 58 mm, 56 mm, 54 mm or 53 mm or less, and preferably 52±0.5 mm.

In an embodiment, the blister pack of the present invention may have an overall length of the receiving part in the B-axis longitudinal direction of 80 to 130 mm, specifically, the lower limit thereof may be 85 mm, 90 mm, or 95 mm or more, and the upper limit thereof may be 125 mm, 120 mm, 115 mm or 110 mm or less, and preferably 100±0.5 mm.

In an embodiment, the blister pack of the present invention may have a length of the receiving part in a direction perpendicular to the B-axis longitudinal direction of 30 to 50 mm, more specifically, the lower limit thereof may be 32 mm, 34 mm, 36 mm, or 37 mm or more, and the upper limit thereof may be 48 mm, 46 mm, 44 mm, 42 mm, or 40 mm or less, and preferably 38±0.5 mm.

The blister pack receiving a syringe of the present invention or capable of receiving a syringe may be shown as shown in FIGS. 1 to 4.

### Syringe Comprising Ophthalmic Formulation

The syringe may comprise:
a body 2 having an outlet 12 at an outlet end 14;
a plunger 4 located inside the body 2; and
a stopper 10 which is located at a front part of the plunger 4 inside the body 2, and includes a front surface 16 forming a variable volume chamber 18 with the body 2 to discharge an ophthalmic formulation 20, which is filled into the variable volume chamber 18, through the outlet 12 by movement of the plunger 4,
wherein the plunger 4 comprises a plunger rod 41 extending in a longitudinal direction, and a flange part 22 located at an end 24 of the plunger rod, which is adjacent to the stopper 10.

In an embodiment, the plunger 4 may include an insertion fixing part 23 in a spherical or threaded shape that protrudes from the flange part 22 toward the stopper 10, and the stopper 10 may be provided with a central recess 62 into which the insertion fixing part 23 is inserted and fixed by press-fit or screw-tightening.

With respect to the insertion fixing part 23, the threaded shape may indicate a threaded shape with a partially protruding surface, and specifically may indicate screw engagement protuberance with a thread forming a helical shape, and as a specific example, may indicate a form such as a screw engagement protuberance-including insertion fixing part 63a as shown on the right side of FIG. 16. The central recess 62 of the stopper 10 may have a screw hole that is threaded 63 so that the insertion fixing part 23 in a threaded shape and the stopper 10 may be engaged in a screw-tightening manner. Here, the screw hole that is threaded 63 may act as a female thread. As shown on the left side of FIG. 16, the insertion fixing part 23 in a threaded shape is represented by the screw engagement protuberance-including insertion fixing part 63a.

In addition, in the insertion fixing part 23, the spherical shape means that, unlike the threaded shape, it does not have a protruding surface such as a screw engagement protuberance and has a smooth shape, and may preferably have a lower end part 64a having a size and shape similar or identical to a bore 64 of the central recess 62 or an upper end part 65a having a size and shape similar or identical to an inner recess 65.

In the syringe, the insertion fixing part 23 is engaged with the central recess 62 of the stopper 10 by a press-fit engagement or a screw-tightening manner, such that the plunger and the stopper are connected by coupling, thereby integrating the plunger and the stopper, which prevents the stopper from being distorted in unpredictable ways due to retraction without harming the sealing and/or sterilization of the final product, allowing for stable storage and proper delivery of the ophthalmic formulation without leakage or contamination.

In an embodiment, the flange part 22 may be arranged to contact the stopper 10, but not couple thereto, which is not engaged in a manner such as a press-fit engagement or a screw-tightening engagement, and the plunger 4 may be used to move the stopper 10 towards the outlet end 14 of the body 2. This movement reduces the volume of the variable volume chamber 18 and causes the fluid within the chamber to be released through the outlet.

In an embodiment, the body 2 of the syringe may be a substantially cylindrical shell, or may include a substantially cylindrical bore with a non-circular outer shape. The outlet end 14 of the body 2 includes an outlet 12 through which the ophthalmic formulation 20 housed within the variable volume chamber 18 may be discharged as the volume of the variable volume chamber is reduced. The outlet 12 may comprise a protrusion from the outlet end 14 through which extends a channel having a smaller diameter than that of the variable volume chamber 18. The outlet may be adapted, for example via a luer lock type connection, for connection to a needle or other accessory such as a sealing device which is able to seal the variable volume chamber, but may be operated, or removed, to unseal the variable chamber and allow connection of the syringe to another accessory, such as a needle. Such a connection may be made directly between the syringe and accessory, or via the sealing device. The body 2 extends along a first axis A from the outlet end 14 to a rear part 25.

The body 2 may include the body part 3, wherein the body part 3 may include an outer peripheral surface area of body part 3a and a thick body part 3b. A wall thickness of the thick body part 3b may be 1.0 to 2.0 mm, preferably 1.2 to 1.8 mm, more preferably 1.45 to 1.65 mm, and most preferably 1.5±0.2 mm.

The wall thickness of the thick body part 3b as described above may minimize the impact when the outer surface of the syringe body is sterilized with gas. Further, it has sufficient advantages of minimizing denaturation reactions, such as oxidation of the active ingredient in the drug solution filled in the syringe body, and facilitating molding.

A length of the body 2 may be 55 to 75 mm, preferably 60 to 70 mm, more preferably 62 to 66 mm, and most preferably 64.4±0.2 mm.

A length of the variable volume chamber 18 may be 45 to 60 mm, preferably 50 to 58 mm, more preferably 52 to 56 mm, and most preferably 53.85±0.2 mm.

The body 2 may be made from a plastic material or from glass, or from any other suitable material and may include indicia on a surface thereof to act as an injection guide. The plastic material may be, for example, a cycloolefin polymer (COP), a cycloolefin copolymer (COC), or the like. Preferably, the plastic material may be COP.

A stopper body 58 of the stopper 10 may be made of rubber, silicone or other suitable resiliently deformable material. According to an embodiment of the present invention, chlorinated butyl rubber is used.

The stopper 10 provides a sealing function by forming a leak-proof seal at the rear of the variable volume chamber 18 to prevent leakage of the ophthalmic formulation 20, which is filled within the variable volume chamber 18. The stopper 10 may be substantially cylindrical and may include one or more circumferential ribs 50, 51, 52 around an outer surface of the stopper, the stopper 10 and ribs 50, 51 being dimensioned such that the ribs 50, 51 may form a substantially formulation-tight seal where the ribs contact the inner surface of the syringe body. The front surface of the stopper 16 may be any suitable shape, for example, may be planar or conical, and preferably conical. Further, the stopper 10 may include the central recess 62 which may be any shape provided the sealing function of the stopper is not compromised. The central recess 62 may be cylindrical in shape or the central recess 62 may include a bore 64 having a first diameter, the bore 64 leading from the rear surface 60 into the inside of the stopper to the inner recess 65 having a second diameter extending longitudinally, the second diameter being larger than the first diameter and conical in shape.

In an embodiment, the stopper may comprise at least two ribs, and more specifically, the stopper may comprise two ribs 51, 52, or three ribs 50, 51, 52.

Furthermore, the central recess 62 may have a screw hole that is threaded 63. Here, the screw hole that is threaded 63 may act as a female thread.

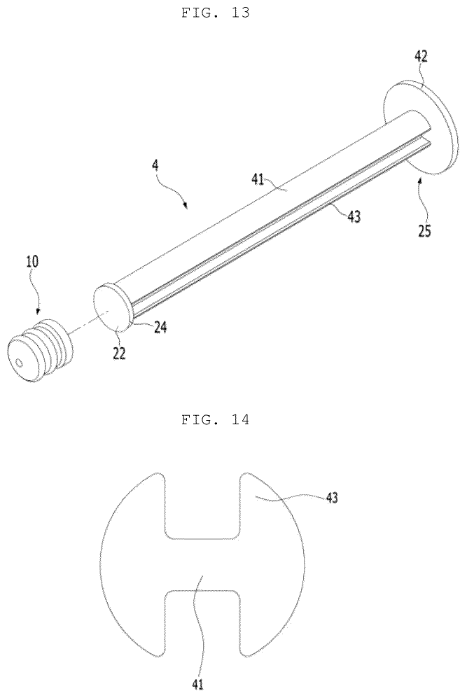

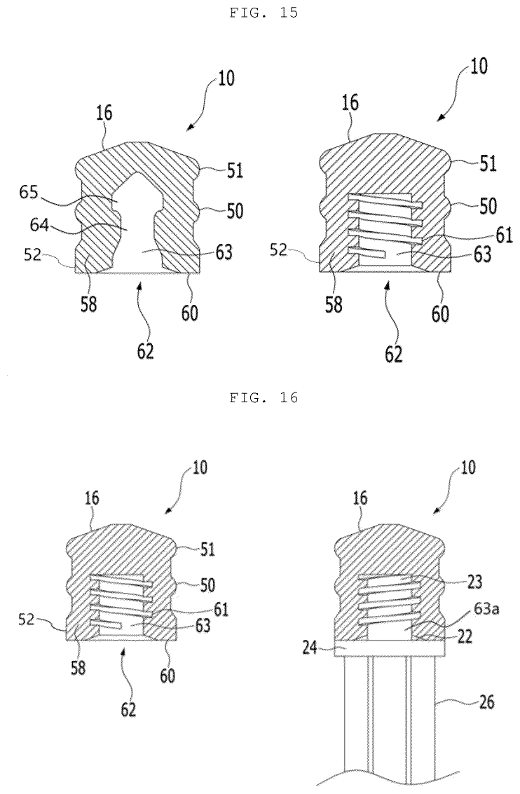

The insertion fixing part 23 of the plunger may be inserted into the central recess 62, thereby coupling the stopper and the plunger. Furthermore, the insertion fixing part 23 may include a spherical or screw-like shape, and may be specifically a threaded (screw) shape or a spherical shape. The insertion fixing part 23 in a spherical-shape may be inserted in a press-fit engagement into the central recess 62 of the resilient stopper and strongly fixed to the stopper by a spherical protrusion of the insertion fixing part 23 (FIG. 14). In addition, the screw engagement protuberance-including insertion fixing part 63a may be strongly fixed to the stopper while being inserted in a threaded engagement manner with the screw hole 63 acting as a female thread (FIG. 13). Additionally, the insertion fixing part 23 of the plunger may be inserted into and coupled to the central recess 62 of the stopper as the removal of material from the central part of the stopper (if this is not necessary for the stopper to perform its required function) reduces the weight of the stopper and reduces the amount of material required to manufacture the stopper.

Further, the plunger that does not include the insertion fixing part 23 may be arranged to contact but not to be coupled with the stopper.

The plunger 4 includes the flange part 22 and the plunger rod 41 extending from the flange part 22 to the rear part 25. The rear part 25 may comprise the disk-shaped flange 42 that is a user-contact portion adapted to be in contact with the user while the formulation filled in the syringe is injected. The disk-shaped flange 42 may comprise a portion that is shaped like a disc, the radius of the disc extending substantially perpendicular to the axis along which the rod extends. The user contact portion could be any suitable shape. The axis along which the plunger rod extends may be the first axis A, or may be substantially parallel with the first axis.

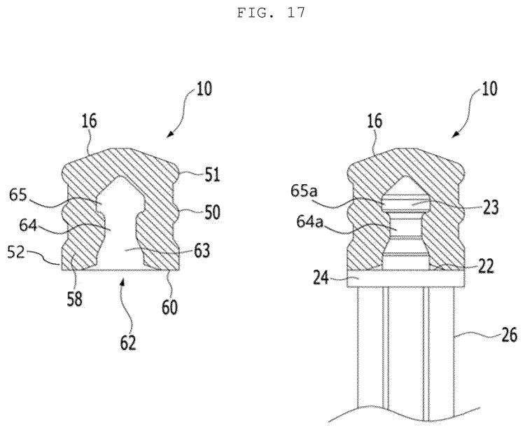

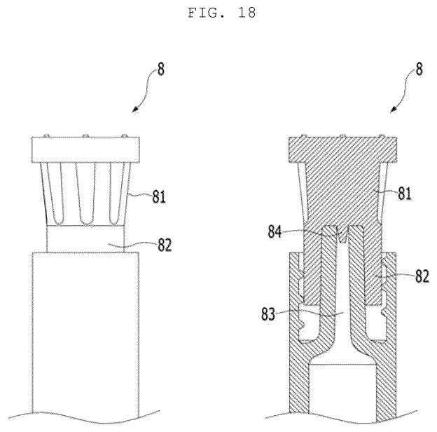

In an embodiment, the flange part 22 may be located at an end adjacent to the stopper 10 and may have a structure that is couplable to the stopper 10. More particularly, by including the insertion fixing part 23 in a spherical or screw shape that protrudes from a central portion of the flange part 22 towards the stopper, the insertion fixing part 23 being inserted in a press-fit manner into the central recess 62 of the stopper, so that the plunger 4 and the stopper 10 may be coupled, or the insertion fixing part 23 being engaged and inserted in a threaded engagement manner into the screw hole 63 in the central recess 62 of the stopper, so that the plunger 4 and the stopper 10 may be coupled, wherein the coupled form is as shown in FIGS. 16 and 17 (right sides of FIGS. 16 and 17, respectively). Further, the flange part 22 may have an outer diameter smaller than the inner diameter of the body 2 and may be circular in shape. Further, a diameter of the flange part 22 may be the same as a diameter of the rear surface 60 of the stopper in contact with the flange part 22. In addition, the flange part 22 may be rotationally symmetrical to the rear surface 60 of the stopper. In the present invention, since the stopper 10 and the plunger 4 may be coupled and mechanically connected, the plunger and the stopper form an integral unit, and thus the stopper may be moved together to the rearward portion of the syringe according to the movement of the plunger. This integration of the stopper and plunger has the advantage of preventing air bubbles generated during the filling process from expanding and causing the stopper to retract under the conditions of reduced pressure that accompany subsequent vapor hydrogen peroxide sterilization or ethylene oxide gas sterilization, allowing the stopper to be controlled so that it no longer retracts from the more precisely intended position, thus avoiding problems such as unexpected sterilization breaks.

In another embodiment, the flange part 22 may be located at an end adjacent to the stopper 10 and may have a structure arranged to contact the stopper 10 but not be coupled thereto.

The plunger rod 41 may have a circular, H-shaped, or cross-sectional shape. The H-shaped cross-section may be formed from a plunger rib 43 centered on the plunger rod 41, as shown in FIG. 6. The plunger rib 43 may extend substantially parallel to an axis along which the plunger rod 41 extends, and the H-shaped cross-section may provide rigidity to the plunger rod 41 without increasing manufacturing complexity. Further, the plunger rod 41 may be made of plastic, such as cycloolefin polymer (COP) and cycloolefin copolymer (COC), preferably made of cycloolefin polymer (COP). The plunger rod 41 may have substantial rigidity under expected conditions of use.

The syringe may comprise the finger grip part 6 arranged on the rear part 25 of the body. The finger grip part 6 may be removable from the syringe. When the body 2 of the syringe includes a terminal flange 28 at an end opposite the outlet end 14, the finger grip part 6 may be configured such that the terminal flange 28 of the body is sandwiched with the sandwich portion 30, which may prevent movement of the finger grip part 6 in a direction parallel to the first axis A.

In addition, the finger grip part 6 may be attached to the body 2 by coupling to a terminal flange 28 of the body 2. The finger grip part 6 may include a sandwich portion 30 which is adapted to substantially sandwich at least some of the terminal flange 28 of the body 2 (FIG. 19).

Further, the finger grip part 6 may include two finger protrusions 40 which extend in opposite directions away from the body 2 perpendicular to the first axis A to facilitate use and handling of the syringe 1.

As noted above, the syringe may be sterilized using a final sterilization method, which is a method of sterilizing the complete article already packaged in its packaging, and these methods may utilize methods known in the art, such as ethylene oxide or vapor hydrogen peroxide sterilization methods.

The problem with this sterilization process is that air bubbles generated during the filling process expand under the reduced pressure that accompanies the sterilization process, causing the stopper and/or plunger rod of the syringe to move within the packaging, resulting in damage by the movement. In connection with the problem, the blister pack for packaging a pre-filled syringe according to the present invention solved the problem of conventional packaging container for pre-filled syringe.

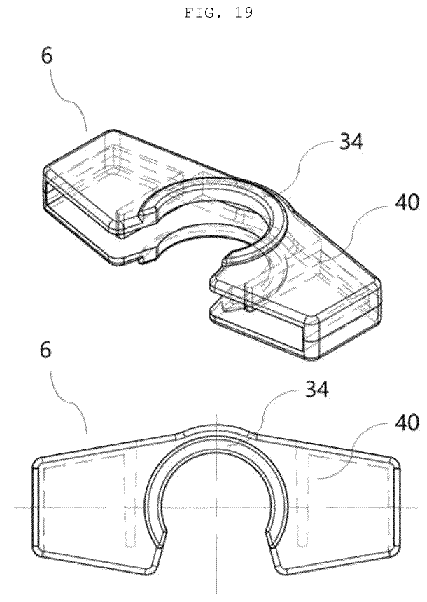

The seal part 8 comprises an upper end part 81 and a lower end part 82, wherein the lower end part may have a diameter smaller than the diameter of the upper end part, and the lower end part 82 may have a recess 83, which is a hollow space with a depression in the center. Into the recess 83, the outlet 12 provided at the outlet end 14 of the body 2 may be inserted, wherein an end of the outlet may contact and seal with a protrusion 84 to prevent the formulation 20 filled in the syringe from leaking out of the outlet 12. Further, the lower end part 82 may include a threaded shape as a portion capable of being engaged with the body 2 so as to be sealed by a screw-tightening action. Accordingly, a portion of the body 2 coupled to the lower end part may also include a threaded shape. The upper end part 81 of the seal part 8 is a user contact part to release the seal caused by the engagement of the lower end part with the body, and may have a shape that repeatedly protrudes at regular intervals to prevent slipping (FIG. 18).

In the present invention, the syringe is suitable for ophthalmic injection and thus has a suitably small volume. The syringe may be configured for ophthalmic injection.

Further, the syringe may contain silicone oil.

In an embodiment, the body 2 includes a silicone film 29 formed by including the body part 3 and applying silicone oil to an inner peripheral surface area 3c of the body, which is the inner wall of the body. Further, the stopper of the present invention may contain silicone oil. As used herein, the expression of containing silicone oil may mean that the surface of the stopper is coated with silicone oil.

The silicone film may be formed by applying silicone in the range of approximately 0.3 mg, 0.4 mg, 0.5 mg, 0.6 mg, 0.7 mg, 0.8 mg, 0.9 mg, 1.0 mg, 1.2 mg, 1.4 mg, 1.5 mg to the inner peripheral surface area 3c of the body part. More specifically, the silicone oil may be applied in an amount of about 0.3 to 1.5 mg, 0.4 to 1.4 mg, more preferably 0.5 to 1.3 mg, and even more preferably 0.6 to 1.2 mg. The application of silicone may minimize denaturation of the drug solution by contact with the inner peripheral surface area 3c of the body part or affecting the efficacy of the drug, and inhibit smearing in the inner peripheral surface area of the body part, while providing sufficient sliding properties to facilitate smooth movement of the stopper.

Further, the syringe may not contain silicone oil.

Specifically, the body and/or stopper of the syringe may not have a silicone oil coating, or there may be no oil present in it. The absence of oil may prevent the possibility of silicone oil being injected into the eye.

The syringe of the present invention is an ophthalmic syringe in which the variable volume chamber 18 may be filled with an injectable ophthalmic formulation and the outlet may be reversibly sealed.

In an embodiment, the variable volume chamber of the syringe may be filled with any suitable injectable liquid or medicament, for example, it may be filled with an injectable medicament. The variable volume chamber has an inner diameter of 5 mm or 6 mm or more and less than 3 mm or 4 mm. The inner diameter may be from 3 mm to 6 mm, or from 4 mm to 5 mm. Further, the volume of the liquid formulation that may be filled in the variable volume chamber is from about 0.05 ml to about 1 ml, preferably from about 0.1 ml to about 0.5 ml, more preferably from 0.14 ml to 0.3 ml, and most preferably from 0.15 ml to 0.2 ml.

Those skilled in the art know that syringes are typically filled to a volume greater than the volume intended for actual administration to the patient, taking into account losses in the manufacture of the injectable syringe. Thus, the actual volume administered to the patient is from 0.01 ml to 1 ml, preferably from 0.02 to 0.5 ml, even more preferably from 0.025 to 0.3 ml, and even more preferably from 0.03 to 0.1 ml.

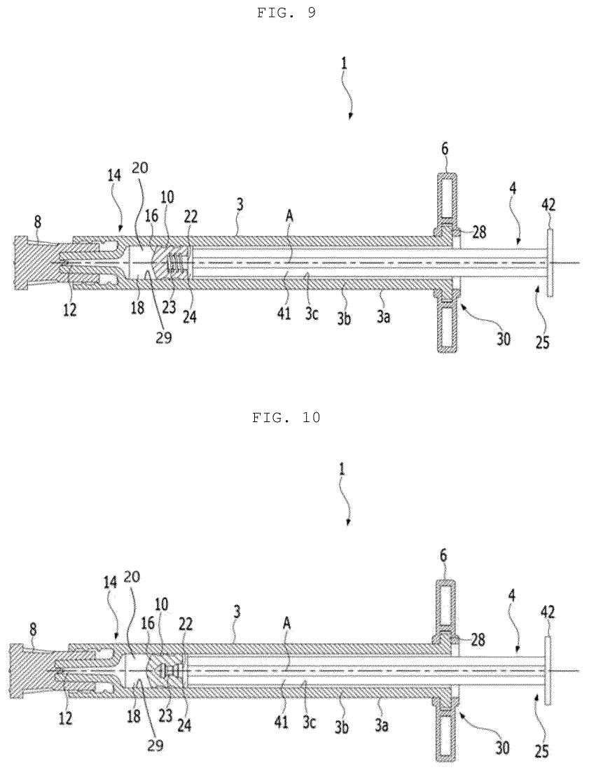

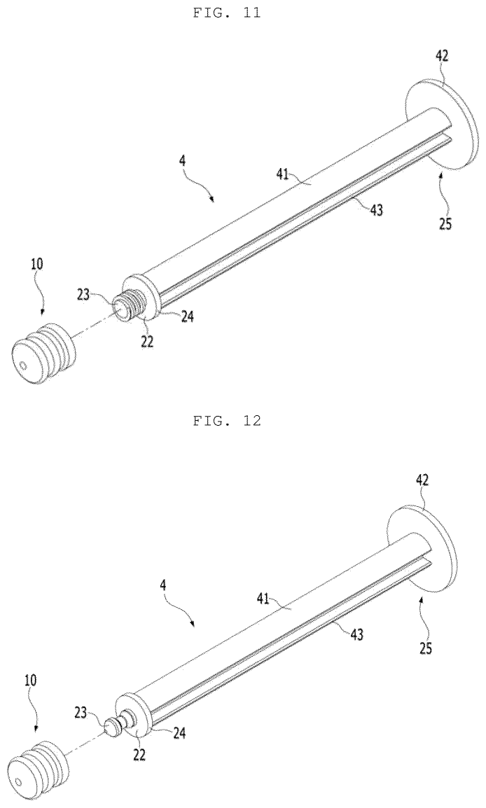

The syringe of the present invention may be illustrated as shown in FIGS. 8 to 10, the plunger as shown in FIGS. 11 to 14, the stopper as shown in FIGS. 15 to 17, the seal part as shown in FIG. 18, and the finger grip part as shown in FIG. 19.

### Ophthalmic Formulation Filled in Syringe

The ophthalmic formulation of the present invention may be filled in the variable volume chamber of the syringe.

The ophthalmic formulation according to the invention is a formulation that is particularly suitable for use under the above-described syringe conditions. The ophthalmic formulation according to the present invention is a formulation capable of retaining the efficacy of the drug over a long period of storage (in particular, minimizing adsorption of the active ingredient to the inner wall or degradation) and securing good stability within the syringe, and may exhibit excellent properties when provided with the above syringe.

The ophthalmic formulation may comprise a therapeutically effective amount of Aflibercept in an aqueous medium.

More specifically, the ophthalmic formulation may comprise, in an aqueous medium, (a) a therapeutically effective amount of Aflibercept; and (b) a stabilizing agent.

More specifically, the ophthalmic formulation may comprise, in an aqueous medium, (a) a therapeutically effective amount of Aflibercept; (b) a stabilizing agent; (c) a surfactant; and (d) an ionic isotonic agent; and may have a pH of 5.2 to 6.2.

In some cases, the ophthalmic formulation may not contain a surfactant or an ionic isotonic agent.

Preferably, the ophthalmic formulation of the present invention may comprise (a) Aflibercept in a therapeutically effective amount of 10 to 100 mg/ml; (b) 2 to 10 w/v % of stabilizing agent; (c) 0.01 to 0.1 w/v % of surfactant; and (d) 5 to 100 mM of ionic isotonic agent; and have a pH of 5.2 to 6.2.

In the present invention, Aflibercept is a VEGF-specific fusion protein in which a VEGF binding site, which is derived from the extracellular domains of human VEGF receptor 1 and VEGF receptor 2, is fused with an Fc region of human IgG1. Specifically, the VEGF-specific fusion protein is a fusion protein in which a region essentially comprising immunoglobulin-like (Ig) domain 2 of human VEGF receptor 1 (Flt1) and Ig domain 3 of human VEGF receptor 2 (Flk1) is fused with the Fc region of human IgG1, and may be Aflibercept having an amino acid sequence of SEQ ID NO: 1 below.

The amino acid sequence of Aflibercept (SEQ ID NO: 1) is shown below:

SDTGRPFVEM YESIPEIIHM TEGRELVIPC TVTSPNITVT LKKFPLDTLI PDGKRIIWDS
RKGFIISNAT YKEIGLLTCE ATVNGHLYKT NYLTHRQTNT IIDVVLSPSH GIELSVGEKL
VLNCTARTEL NVGIDFNWEY PSSKHQHKKL VNRDLKTQSG SEMKKFLSTL TIDGVTRSDQ
GLYTCAASSG LMTKKNSTFV RVHEKDKTHT CPPCPAPELL GGPSVFLFPP KPKDTLMISR
TPEVTCVVVD VSHEDPEVKF NWYVDGVEVH NAKTKPREEQ YNSTYRVVSV LTVLHQDWLN
GKEYKCKVSN KALPAPIEKT ISKAKGQPRE PQVYLTPPSR DELTKNQVSL TCLVKGFYPS
DIAVEWESNG QPENNYKTTP PVLDSDGSFF LYSKLTVDKS RWQQGNVFSC SVMHEALHNH
YTQKSLSLSP G

(Disulfide bridge: 30-79; 124-185; 246-306; 352-410, Dimer: 211; 214)

Aflibercept may be recombinantly or synthetically produced.

As used herein, “stable” or “remains stable” may mean that the structure and/or physical, chemical, and/or biological properties of the proteins in the composition are maintained during storage (e.g., low protein dimer, multimer, polymer formation rates, low protein aggregation rates, low protein fragmentation rates, low degradation rates, and/or low denaturation rates during storage). Various analytical techniques for measuring protein stability are well known in the art. When observing proteins by visual inspection of its color and/or transparency, or measuring them by UV light scattering (which measures visible aggregates) or by size exclusion chromatography (SEC), a case where the protein shows little or no change in aggregation, precipitation and/or denaturation may be understood to “maintain its physical stability.”

The ophthalmic formulation of the present invention may comprise Aflibercept in a therapeutically effective amount of 10 to 100 mg/ml, preferably in an amount of 10 to 50 mg/ml, more preferably in an amount of 20 to 50 mg/ml.

In the present invention, the ophthalmic formulation may further comprise a buffer solution of 5 to 20 mM, wherein a concentration of the buffer solution may be preferably 7 to 15 mM, more preferably 8 to 12 mM. The “buffer solution” refers to a buffer solution that resists a change in pH due to the action of its acid-base conjugate component. In an embodiment of the present invention, the buffer solution may be an acetate buffer solution, preferably comprising a sodium acetate buffer solution.

The aqueous medium includes, without limitation, any aqueous medium capable of providing a buffer together with the acetate salt buffer, including, for example, distilled water for injection, sterile purified water, and the like.

In the present invention, the stabilizing agent may comprise sucrose, trehalose, mannitol, glucose, and the like, preferably sucrose. Further, a concentration of the stabilizing agent may be from 2 to 12 w/v %, preferably from 3 to 10 w/v %, more preferably from 5 to 8 w/v %.

In the present invention, the surfactant may comprise polysorbate 20, polysorbate 40, polysorbate 60, polysorbate 80, and the like, and preferably polysorbate 20 may be used. Further, the formulation may comprise the surfactant in a concentration of 0.01 to 0.1 w/v %, preferably 0.01 to 0.08 w/v %, more preferably 0.01 to 0.05 w/v %.

In the present invention, the ionic isotonic agent is sodium chloride, and the formulation of the present invention may comprise sodium chloride, as the ionic isotonic agent, at a concentration of 5 to 100 mM, preferably 10 to 50 mM, more preferably 10 to 40 mM.

The formulation provided herein may be isotonic with the living body. For example, the formulation may have an osmotic pressure of from about 200 mOsm/kg to about 400 mOsm/kg, such as from about 250 mOsm/kg to about 300 mOsm/kg, and the scope of the present invention is not limited thereto, and the osmotic pressure may be controlled by a stabilizing agent.

The ophthalmic formulation according to the present invention may comprise (a) Aflibercept in a therapeutically effective amount of 20 to 50 mg/ml; (b) 8 to 12 mM of sodium acetate buffer; (c) 5 to 8 w/v % of sucrose; (d) 0.01 to 0.05 w/v % of polysorbate 20; and (e) 10 to 40 mM of an ionic isotonic agent of sodium chloride; and the pH of the formulation may be 5.4 to 6.2, preferably, 5.4 to 6.0.

More specifically, the ophthalmic formulation according to the present invention may comprise (a) Aflibercept in a therapeutically effective amount of about 40 mg/ml; (b) about 10 mM of sodium acetate buffer; (c) about 6.5 w/v % of sucrose; (d) 0.03 w/v % of polysorbate 20; and (e) about 15 mM of an ionic isotonic agent of sodium chloride; and the pH of the formulation may be 5.4 to 6.0. Specifically, the pH of the formulation may be about 5.4, 5.5, 5.6, 5.7, 5.8, 5.9, or 6.0.

The syringe of the present invention in the ophthalmic formulation may be provided in the form of a pre-filled syringe.

The ophthalmic formulation according to the present invention has good stability and exhibits good inhibition of the formation of aggregates (dimer, multimer, etc.) and fragments.

In another aspect of the invention, the present invention also provides a kit comprising one or more pre-filled syringes of the present invention. Preferably, the kit comprises a blister pack. The kit may further comprise a needle if the pre-filled syringe does not include a stack-in needle. The kit may further comprise an instruction manual.

From the above description, those skilled in the art to which the present disclosure pertains will understand that the present disclosure may be embodied in other specific forms without changing the technical spirit or essential characteristics thereof. In this regard, it should be understood that the embodiments described above are illustrative in all respects and not restrictive. As the scope of the present disclosure, it should be construed that all changes or modifications derived from the meaning and scope of the claims to be described below and equivalents thereof rather than the above detailed description are included in the scope of the present disclosure.

[Detailed Description of Main Elements]

| | |
|---|---|
| 1: Syringe | 2: Body |
| 3: Body part | 3a: Outer peripheral |
| | surface area of |
| | body part |
| 3b: Thick body part | 3c: Inner peripheral |
| | surface area of |
| | body part |
| 4: Plunger | 6: Finger grip part |
| 8: Seal part | A: First axis |
| 10: Stopper | 12: Outlet |
| 14: Outlet end | 16: Front surface |
| 18: Variable volume chamber | 20: Ophthalmic formulation |
| 22: Flange part | 23: Insertion fixing part |
| 24: Plunger end | 25: Rear part |
| 28: Terminal flange | 29: Silicone membrane |
| 30: Sandwich portion | 34: Grip protrusion |
| 36: Boer | 38: Rear end |
| 40: Finger protrusion | 41: Plunger rod |
| 42: Disc-shaped flange | 43: Plunger rib |
| 46: Disk-shaped portion | 50, 51, 52: Ribs |
| 58: Stopper body | 60: Rear surface |
| 61: Thread line of screw hole | |
| 62: Central recess | |
| 63: Screw hole that is threaded | |
| 63a: Screw engagement protuberance- | |
| including insertion fixing part | |
| 64: Bore having first diameter | |
| 65: Inner recess having second diameter | |
| 64a: Lower end part of insertion | |
| fixing part in spherical shape | |
| 65a: Upper end part of insertion | |
| fixing part in spherical shape | |
| 81: Upper end part | 82: Lower end part |
| 83: Recess | 84: Protrusion |
| 7: Blister pack | 71: Container body |
| 72: Receiving part | 72a: First receiving part |
| 72b: Second receiving part | 72c: Third receiving part |
| 73: Sealing part | 74: Sealing film |
| 75: Fixing part | 75a: First fixing part |
| 75b: Second fixing part | B: Second axis |

1. A blister pack of a syringe, comprising:
a container body including a receiving part containing a space molded to accommodate a pre-filled syringe;
a fixing part protruding to fix a portion of the syringe accommodated in the receiving part; and
a sealing part to which a sealing film is attachable,
wherein the fixing part includes a second fixing part and optionally further includes a first fixing part,
the receiving part is separated by the fixing part to form at least any one of a first receiving part and a second receiving part, and a third receiving part, and
when the syringe is accommodated in the receiving part of the blister pack, a movable distance of a plunger of the syringe is less than or equal to a spacing distance between ribs included in a stopper.

2. The blister pack of a syringe of claim 1, wherein the fixing part comprises the first fixing part and the second fixing part, and the receiving part comprises the first receiving part and the second receiving part separately formed by the first fixing part, and comprises the second receiving part and the third receiving part separately formed by the second fixing part.

3. The blister pack of a syringe of claim 1, wherein the fixing part comprises the second fixing part, and comprises the second receiving part and the third receiving part separately formed by the second fixing part.

4. The blister pack of a syringe of claim 1, wherein the spacing distance between the ribs included in the stopper is 1 to 4 mm.

5. The blister pack of a syringe of claim 1, wherein the container body constituting the blister pack is formed of a plastic film or sheet with rigid properties in order to fix and maintain the accommodated syringe.

6. The blister pack of a syringe of claim 1, wherein the container body is formed of one or more plastic films or sheets selected from the group consisting of polyvinyl chloride (PVC), polyethylene terephthalate (PET), polypropylene (PP), polystyrene, polyethylene, polyester, and polypropylene.

7. The blister pack of a syringe of claim 1, wherein the syringe is accommodated in the receiving part and a portion of the body is interlocked by the fixing part, and the fixing part protrudes in an inner direction of the container body so that the portion of the body of the syringe is fixed in a tightening manner by the fixing part.

8. The blister pack of a syringe of claim 2, wherein the first receiving part receives a portion of an upper part of the syringe, including a seal part of the syringe, the second receiving part receives a central part of the syringe, including the portion of the body of the syringe, and the third receiving part receives a rear part of the plunger, including a finger grip part and a disk-shaped flange of the syringe.

9. The blister pack of a syringe of claim 3, wherein the second receiving part receives an upper part of the syringe, including a seal part of the syringe, and a central part of the syringe, including a portion of the body of the syringe, and the third receiving part receives a rear part of the plunger, including a finger grip part and a disk-shaped flange of the syringe.

10. The blister pack of a syringe of claim 1, wherein a wall of the third receiving part facing the disk-shaped flange supports the disk-shaped flange of the plunger, thereby preventing the syringe or plunger from retracting and moving in a second axis longitudinal direction.

11. The blister pack of a syringe of claim 1, wherein the disc-shaped flange is in contact with a wall of the third receiving part facing the disc-shaped flange.

12. The blister pack of a syringe of claim 1, wherein the syringe does not include a plunger shoulder.

13. The blister pack of a syringe of claim 1, wherein the blister pack comprises a sealing film adhered to the sealing part, and
the receiving part is sealed by adhesion of the sealing part to the sealing film.

14. The blister pack of a syringe of claim 1, wherein the sealing part comprises an adhesive member so that one side of the sealing film is attached.

15. The blister pack of a syringe of claim 1, wherein the sealing film is detachably adhered to the sealing part.

16. The blister pack of a syringe of claim 1, wherein the syringe comprises:
a body having an outlet at an outlet end;
a plunger located inside the body; and
a stopper which is located at a front part of the plunger inside the body, and includes a front surface forming a variable volume chamber with the body to discharge an ophthalmic formulation, which is filled into the variable volume chamber, through the outlet by movement of the plunger,
wherein the plunger comprises a plunger rod extending in a longitudinal direction, and a flange part located at an end of the plunger rod, which is adjacent to the stopper.

17. The blister pack of a syringe of claim 1, wherein the syringe comprises an ophthalmic formulation.

18. The blister pack of a syringe of claim 17, wherein the ophthalmic formulation is used to administer to patients with ophthalmic diseases.


SEQUENCE LISTING (1 sequence)

SEQ ID NO: 1 - protein, 431 aa, synthetic construct
  REGION 1..431: note = Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG